(19) United States
(12) Patent Application Publication
(10) Pub. No.: US 2024/0115873 A1
(43) Pub. Date: Apr. 11, 2024

(54) ULTRAVIOLET THERAPY APPARATUS AND METHOD FOR APPLYING ULTRAVIOLET LIGHT USING ULTRAVIOLET THERAPY APPARATUS

(71) Applicants: PUBLIC UNIVERSITY CORPORATION NAGOYA CITY UNIVERSITY, Nagoya-city, Aichi (JP); USHIO DENKI KABUSHIKI KAISHA, Tokyo (JP)

(72) Inventors: Akimichi MORITA, Aichi (JP); Takashi HORIO, Tokyo (JP); Tomohiko KIO, Tokyo (JP); Hiroshi SHIBATA, Tokyo (JP)

(73) Assignees: PUBLIC UNIVERSITY CORPORATION NAGOYA CITY UNIVERSITY, Nagoya-city, Aichi (JP); USHIO DENKI KABUSHIKI KAISHA, Tokyo (JP)

(21) Appl. No.: 18/257,562

(22) PCT Filed: Dec. 9, 2021
(86) PCT No.: PCT/JP2021/045410

§ 371 Date: Jun. 14, 2023

(30) Foreign Application Priority Data
Dec. 14, 2020 (JP) 2020-206478

(51) Int. Cl. A61N 5/06

(52) U.S. Cl. CPC A61N 5/06; A61N 2005/0628; A61N 2005/0651; A61N 2005/0661

(57) ABSTRACT

The ultraviolet therapy apparatus includes a light source part having an LED light source, a controller that controls lighting of the LED light source, an input unit that inputs a set irradiation dose of a therapeutic light to be applied to a patient on the assumption that a reference light is used for the therapeutic light, a storage unit that stores at least one parameter for modifying the set irradiation dose. The controller includes a modifier that modifies the set irradiation dose on the basis of the parameter stored in the storage unit, and a lighting controller that causes the LED light source to provide the modified irradiation dose. The modified irradiation dose is derived from a degree of effect of the reference light on a human body and a degree of effect on the human body caused by the ultraviolet light output from the light source part.

### TECHNICAL FIELD

The present invention relates to ultraviolet therapy apparatuses that use LEDs for light sources and methods for applying ultraviolet light using the ultraviolet therapy apparatuses.

### BACKGROUND ART

Conventional phototherapy includes ultraviolet therapy that uses ultraviolet light in the wavelength range of UVA (wavelength 320 nm to 400 nm) and UVB (wavelength 280 nm to 320 nm). Ultraviolet therapy is a treatment that uses ultraviolet light irradiation to achieve therapeutic effects for immunosuppression.

For example, Patent Document 1 (JP-A-2017-131522) discloses an ultraviolet therapy apparatus that treats skin diseases with ultraviolet light. This ultraviolet therapy apparatus includes a light source lamp or a LED as a source of ultraviolet light.

In a case in which LEDs are used for the light source, the circuit configuration therefor can be generally simpler than that for the power supply for the lamp, and the apparatus can be made smaller and lighter. For this reason, there have been proposed ultraviolet therapy apparatuses that use ultraviolet light emitting devices (UV LEDs) as the light sources for ultraviolet light.

In the following description, ultraviolet light and light containing ultraviolet light are sometimes simply referred to as “light.”

### BACKGROUND DOCUMENT

### Patent Document

Patent Document 1: JP-A-2017-131522

### SUMMARY OF THE INVENTION

### Problem to be Solved by the Invention

For example, an ultraviolet therapy apparatus that uses an excimer lamp that emits ultraviolet light within the wavelength range of UVB having a peak at a wavelength of 308 nm is known.

On the other hand, there is an LED that emits ultraviolet light with a peak at a wavelength of 308 nm (hereinafter referred to as “308 nm LED”). Accordingly, it is conceivable to adopt a therapy apparatus that uses a 308 nm LED instead of the UVB therapy apparatus that uses the above-mentioned excimer lamp.

However, in contrast to lamps, the peak wavelength of light emitted from LEDs may vary in a range from about minus 5 nm to about plus 5 nm due to manufacturing variations although the LEDs are designed for the peak wavelength of 308 nm. In other words, 308 nm LEDs include not only those with a peak wavelength at 308 nm, but also those with a peak wavelength in the range of 303 nm to 313 nm.

The effects of ultraviolet rays in the range of UVB on the skin differ depending on the wavelength. Generally, in ultraviolet therapy apparatuses, affected areas are irradiated with light with an appropriate dose that do not cause a side effect in order to obtain a desired therapeutic effect. The appropriate dose depends on the wavelength of the light emitted from the light source.

If the wavelengths of the light emitted from the ultraviolet therapy apparatus vary depending on the individual LEDs used for the light sources, a side effect may occur or, conversely, the therapeutic effect may be insufficient even if affected areas are irradiated with the light with a dose that is intended not cause a side effect.

Accordingly, it is an object of the present invention to provide an ultraviolet therapy apparatus that can obtain excellent therapeutic effects without causing a side effect regardless of individual variation among LED light sources, and to provide a method for applying ultraviolet light by the ultraviolet therapy apparatus.

### Means for Solving the Problem

In accordance with an aspect of the present invention, there is provided an ultraviolet therapy apparatus, including a light source part having an LED light source that emits light including ultraviolet light and a light-outputting surface through which the light from the LED light source is output; a controller configured to control lighting of the LED light source; an input unit configured to input a set irradiation dose, which is an irradiation amount of a therapeutic light to be applied to a patient on the assumption that a reference light emitted from a reference light source is used for the therapeutic light; and a storage unit for storing at least one parameter for modifying the set irradiation dose. The controller includes a modifier configured to modify the set irradiation dose input by the input unit on the basis of the parameter stored in the storage unit to obtain a modified set irradiation dose of the ultraviolet light, and a lighting controller configured to cause the LED light source to provide the modified set irradiation dose of the ultraviolet light obtained by the modifier. The modified set irradiation dose is derived from a degree of effect of the reference light on a human body and a degree of effect on the human body caused by the ultraviolet light output from the light-outputting surface.

Thus, the set irradiation dose, which is suitable if the reference light is used for the therapeutic light, is modified on the basis of the degree of effect of the reference light on the human body and the degree of effect on the human body caused by the ultraviolet light output from the light-outputting surface of the ultraviolet therapy apparatus (subject therapy apparatus), which is actually used for therapeutic treatment, and the LED light source is controlled to provide the modified set irradiation dose. Accordingly, if there is a difference in wavelength between the reference light and the light emitted from the therapy apparatus, ultraviolet irradiation can be applied with an irradiation dose that is suitable for the light emitted from the therapy apparatus. Therefore, excellent therapeutic effects can be obtained without causing a side effect to the affected area.

In the above ultraviolet therapy apparatus, the storage unit may store, as the at least one parameter, a modification factor, which is a value obtained by dividing the degree of effect of the reference light on the human body by the degree of effect on the human body caused by the ultraviolet light output from the light-outputting surface, and the modifier may be configured to calculate the modified set irradiation dose by multiplying the set irradiation dose input by the input unit by the modification factor stored in the storage unit.

In this case, the modified set irradiation dose can be derived by a simple calculation.

In the above ultraviolet therapy apparatus, the storage unit may store, as the at least one parameter, at least a spectral spectrum of the reference light, a spectral spectrum of the ultraviolet light output from the light-outputting surface, and an erythema action spectrum. The modifier may be configured to calculate the degree of effect of the reference light on the human body on the basis of a product of a relative irradiation intensity of the spectral spectrum of the reference light and a relative effectiveness of the erythema action spectrum stored in the storage unit, calculate the degree of effect on the human body caused by the ultraviolet light output from the light-outputting surface on the basis of a product of the spectral spectrum of the ultraviolet light output from the light-outputting surface and the erythema action spectrum stored in the storage unit, calculate a modification factor, which is a value obtained by dividing the degree of effect of the reference light on the human body by the degree of effect on the human body caused by the ultraviolet light output from the light-outputting surface, and calculate the modified set irradiation dose by multiplying the set irradiation dose input by the input unit by the modification factor.

In this case, the modified set irradiation dose can be derived appropriately.

In the above ultraviolet therapy apparatus, the storage unit may store an irradiance of the ultraviolet light output from the light-outputting surface, and the lighting controller may be configured to calculate an irradiation time of the ultraviolet light from the LED light source by dividing the modified set irradiation dose by the irradiance of the ultraviolet light output from the light-outputting surface, and to cause the LED light source to emit the ultraviolet light for the irradiation time.

In this case, the irradiation time of the LED light source can be controlled so that the LED light source can provide the modified set irradiation dose appropriately.

In the above ultraviolet therapy apparatus, the lighting controller may be configured to calculate an irradiance of the ultraviolet light to be output from the light-outputting surface by dividing the modified set irradiation dose by a predetermined irradiation time, and to cause the LED light source to emit the ultraviolet light at the irradiance for the predetermined irradiation time.

In this case, the current value for the LED light source can be controlled so that the LED light source can provide the modified set irradiation dose appropriately.

In the above ultraviolet therapy apparatus, the degree of effect may be an integral value of a product of a relative irradiation intensity of a spectral spectrum and a relative effectiveness of an erythema action spectrum over a predetermined wavelength range.

In this case, the set irradiation dose can be appropriately modified on the basis of the degree of effect on the human body by the reference light and the degree of effect on the human body by the ultraviolet light emitted from the therapy apparatus.

In the above ultraviolet therapy apparatus, the wavelength range may be from 250 nm to 400 nm.

In this case, it is possible to obtain the modified set irradiation dose on the basis of an appropriately derived degree in the wavelength range in which the erythema action spectrum is defined.

In the above ultraviolet therapy apparatus, the degree of effect is a UV index.

In this case, by using a UV index measuring instrument, the degree of effect on the human body can be measured and calculated more simply.

In the above ultraviolet therapy apparatus, the LED light source may be manufactured to emit the ultraviolet light having a peak wavelength within a range of 308 nm to 313 nm.

In this case, various skin diseases to which mid-wavelength ultraviolet therapy is effectively applied can be appropriately treated.

In the above ultraviolet therapy apparatus, the LED light source may be manufactured to emit the ultraviolet light having a peak wavelength at 308 nm.

In this case, various skin diseases can be appropriately treated in the same way as that with an ultraviolet therapy apparatus having a conventional excimer lamp light source having a wavelength peak at 308 nm.

The reference light source may be a lamp that emits the reference light having a peak wavelength within a range of 308 nm and 313 nm.

In this case, the irradiation dose that has been set for the conventional ultraviolet therapy apparatus using the lamp for the light source can be used as the set irradiation dose to be input to the therapy apparatus.

The reference light source may be an excimer lamp.

In this case, the irradiation dose that has been set for the conventional ultraviolet therapy apparatus using the excimer lamp for the light source can be used as the set irradiation dose to be input to the therapy apparatus.

The reference light source may be an LED light source that emits the reference light having a peak wavelength within a range of 308 nm and 313 nm.

In this case, the actual irradiation dose when an LED light source having a specific wavelength is used as the light source can be used as the set irradiation dose to be input to the therapy apparatus.

In accordance with an aspect of the present invention, there is provided a method for applying ultraviolet light using an ultraviolet therapy apparatus having a light-outputting surface through which light including ultraviolet light emitted from an LED light source is output. The method includes a first step of inputting a set irradiation dose, which is an irradiation amount of a therapeutic light to be applied to a patient on the assumption that a reference light emitted from a reference light source is used for the therapeutic light; a second step of modifying the set irradiation dose by comparing a degree of effect on a human body by the reference light with a degree of effect on the human body by the ultraviolet light output from the light-outputting surface to obtain a modified set irradiation dose of the ultraviolet light; and a third step of causing the LED light source to provide the modified set irradiation dose.

Thus, the set irradiation dose, which is suitable if the reference light is used for the therapeutic light, is modified on the basis of the degree of effect of the reference light on the human body and the degree of effect on the human body caused by the ultraviolet light output from the light-outputting surface of the ultraviolet therapy apparatus, which is actually used for therapeutic treatment, and the LED light source is controlled to provide the modified set irradiation dose. Accordingly, if there is a difference in wavelength between the reference light and the light emitted from the therapy apparatus, ultraviolet irradiation can be applied with an irradiation dose that is suitable for the light emitted from the therapy apparatus. Therefore, excellent therapeutic effects can be obtained without causing a side effect to the affected area.

In the method for applying ultraviolet light, the second step may include a step of measuring a spectral spectrum of the ultraviolet light output from the light-outputting surface in a predetermined wavelength range; a step of calculating an erythemal ultraviolet irradiance by integrating a product of a relative irradiation intensity of the spectral spectrum of the ultraviolet light output from the light-outputting surface and a relative effectiveness of an erythema action spectrum over the wavelength range; a step of calculating a relative erythemal ultraviolet irradiance by normalizing the erythemal ultraviolet irradiance by an integral value of the spectral spectrum of the ultraviolet light output from the light-outputting surface over the wavelength range; a step of measuring a spectral spectrum of the reference light in the wavelength range; a step of calculating a reference erythemal ultraviolet irradiance by integrating a product of a relative irradiation intensity of the spectral spectrum of the reference light and the relative effectiveness of the erythema action spectrum of the reference light and the erythema action spectrum over the wavelength range; a step of calculating a reference relative erythemal ultraviolet irradiance by normalizing the reference erythemal ultraviolet irradiance by an integral value of the spectral spectrum of the reference light over the wavelength range; a step of calculating a modification factor by dividing the reference relative erythemal ultraviolet irradiance by the relative erythemal ultraviolet irradiance; a step of calculating the modified set irradiation dose by multiplying the set irradiation dose by the modification factor.

In this case, the modified set irradiation dose can be calculated appropriately.

### Effects of the Invention

According to the present invention, in an ultraviolet therapy apparatus using an LED (UV LED) light source that emits ultraviolet light as a light source, an excellent therapeutic effect can be obtained without causing a side effects, regardless of individual variation among the LED light sources.

The objects, modes, and effects of the invention described above, as well as those not described above, will be understood by those skilled in the art from the following description of the mode of implementing the invention (detailed description of the invention) by referring to the accompanying drawings and the claims.

### BRIEF DESCRIPTION OF THE DRAWINGS

FIG. 1 is a graph showing the erythema action spectrum defined by the CIE;

FIG. 2 is a graph showing spectral spectra;

FIG. 3 is a graph showing the products of the relative irradiation intensities of the spectral spectra shown in FIG. 2 and the erythema action spectrum shown in FIG. 1;

FIG. 4 is a graph showing a ratio of MED for each individual;

FIG. 5 is a flowchart showing a processing flow in a conventional ultraviolet therapy apparatus;

FIG. 6 is a flowchart showing a processing flow in an ultraviolet therapy apparatus according to an embodiment of the present invention; and

FIG. 7 is a block diagram showing an example configuration of the ultraviolet therapy apparatus.

### DESCRIPTION OF EMBODIMENT

Hereinafter, with reference to the accompanying drawings, an embodiment of the present invention will be described.

As an embodiment, an ultraviolet therapy apparatus that includes a treatment tool that emits light containing ultraviolet light, for example, within the range of UVB (wavelength 280 nm to 320 nm) will be described. The ultraviolet therapy apparatus includes an LED light source manufactured so as to emit light having a peak at a wavelength of 308 nm.

Exposure of human skin to ultraviolet light in the range of UVB causes erythema as a side effect. Erythema is redness of the skin surface caused by dilation of capillaries or other reasons. The dose of ultraviolet light irradiation after which a minimally perceptible skin erythema can be detected is referred to as the minimal erythema dose (MED). The unit of MED is mJ/cm². In precisely the same way as the susceptibility to sunburn varies among individuals, the susceptibility to erythema, or MED varies among individuals.

The susceptibility to ultraviolet erythema, i.e., the degree of effect of ultraviolet on human bodies varies depending on the wavelength of the ultraviolet. The relative effectiveness on human bodies depending on the wavelength is defined by the International Commission on Illumination (CIE) as the erythema reference action spectrum.

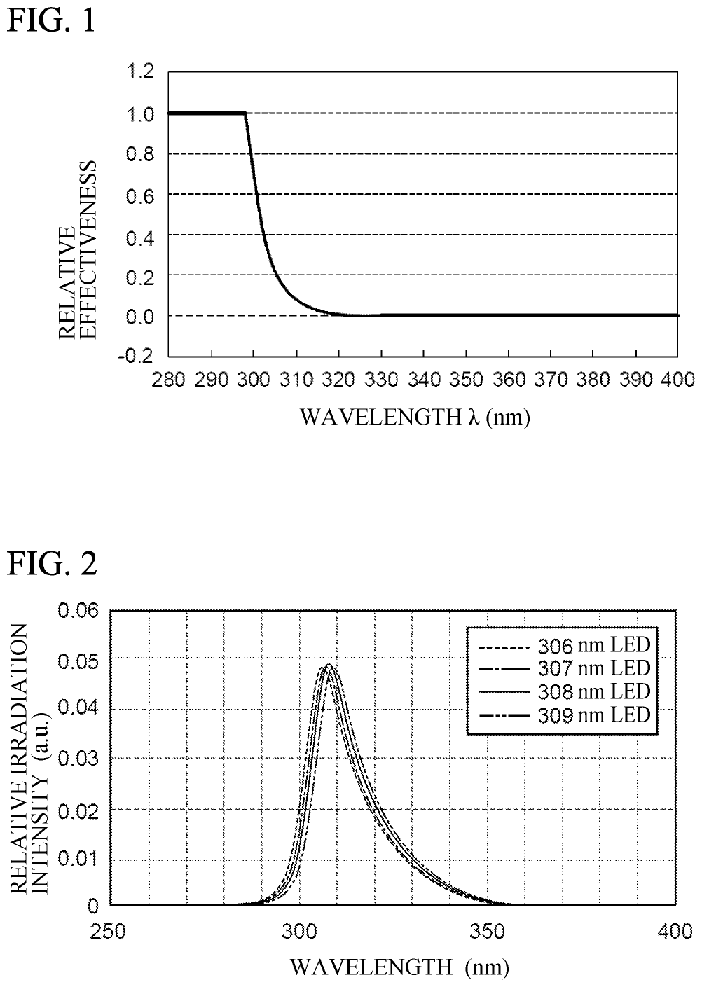

FIG. 1 is a graph showing the erythema action spectrum Sₑᵣ.

In FIG. 1, the horizontal axis indicates the wavelength λ (nm) and the vertical axis indicates a relative effectiveness. The erythema action spectrum Sₑᵣ is defined in the wavelength range of 250 nm to 400 nm, and is defined as a relative effectiveness depending on wavelengths as in Formula (1), in which the relative effectiveness is a relative value on the assumption that the effect of light with a wavelength of 250 nm to 298 nm on the human skin is one.

$${S}_{er} ( λ ) = \{ \begin{matrix}1 & ( when 250 nm < λ < 298 nm ) \\ {10}^{0.094 ( 298 - λ )} & ( when 298 nm \leq λ \leq 328 nm ) \\ {10}^{0.015 ( 139 - λ )} & ( when 328 nm < λ < 400 nm )\end{matrix} \qquad ( 1 )$$

From the outline of the graph shown in FIG. 1, it can be understood that shorter wavelengths have a greater impact on human bodies and are more likely to cause erythema. Specifically, light with wavelengths longer than the UVB region, or longer than the wavelength of 328 nm (in a case in which Formula (1) is strictly applied), has little impact on the skin. On the other hand, light with wavelengths below 328 nm affects the skin, and this effect increases as the wavelength is shorter.

This indicates that MED is less at shorter wavelengths. In other words, MED is inversely related to the erythema action spectrum Sₑᵣ.

The overall effect of ultraviolet radiation on the human body is obtained by integrating the product of the spectral irradiance of the ultraviolet and the erythema action spectrum Sₑᵣ in the wavelength range of 250 nm to 400 nm. The effect thus obtained is called the erythemal ultraviolet irradiance I_{CIE}. The erythemal ultraviolet irradiance ICIE is calculated as in Formula (2).

I_{CIE}=∫₂₅₀⁴⁰⁰E_{λ}×Sₑᵣdλ, (2)

In addition, the UV index I_{UV} is often used as the overall degree of effect of ultraviolet radiation on the human body. The UV index I_{UV} and the erythematic UV dose I_{CIE} are in the relationship of Formula (3). The UV index I_{UV} can be measured by a simple measuring instrument.

I_{UV}=I_{CIE}/25 (3)

As can be seen from FIG. 1, especially in the wavelength range of 298 nm to 310 nm, even only a one nanometer change in the wavelength causes a significant change in the degree of effect on the human body.

FIG. 2 is a graph showing spectral spectra of lights from LEDs with peak wavelengths of 306 nm, 307 nm, 308 nm, and 309 nm. The spectral spectra are normalized so that the integrated value is one in the wavelength range of 250 nm to 400 nm. The value of the vertical axis in FIG. 2 is expressed by Formula (4).

E_{λ}/(∫₂₅₀⁴⁰⁰E_{λ}d_{λ}) (4)

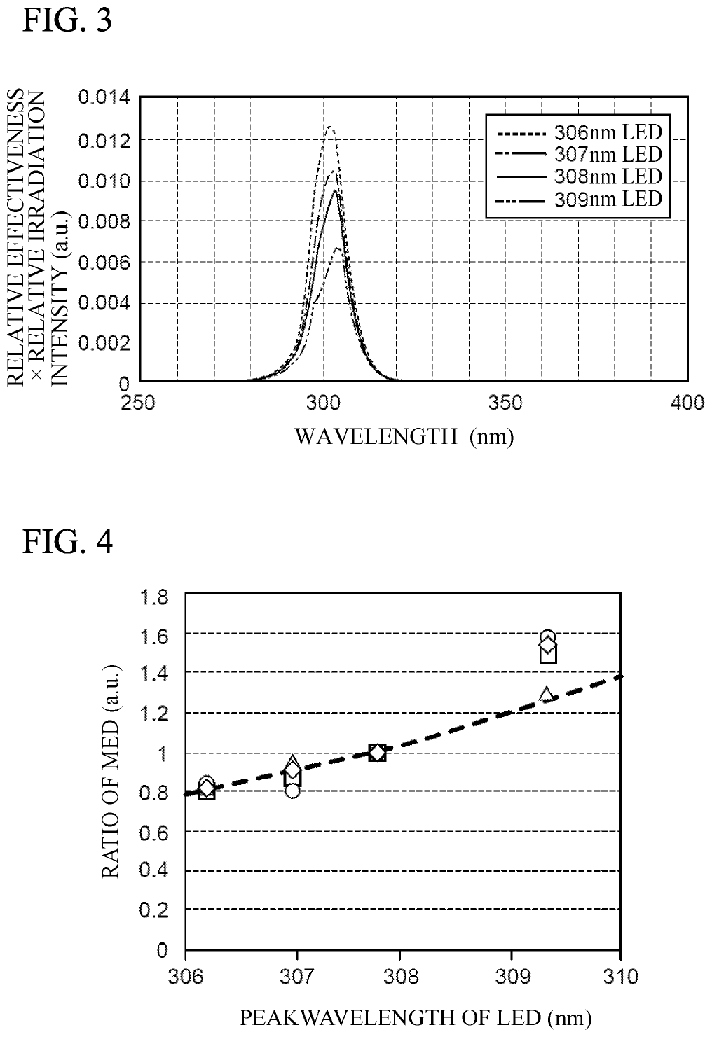

FIG. 3 is a graph showing the products of the relative irradiation intensities of the spectral spectra shown in FIG. 2 and the relative effectiveness of the erythema action spectrum Sₑᵣ shown in FIG. 1. The value of the vertical axis in FIG. 3 is expressed by Formula (5).

E_{λ}×Sₑᵣ/(∫₂₅₀⁴⁰⁰E_{λ}d_{λ}) (5)

As shown in FIG. 3, even only a change in the peak wavelength by 1 nm causes a significant change in the degree of effect on the human body.

Accordingly, for example, when a person tries to undergo therapeutic treatment with an ultraviolet therapy apparatus that uses a 308 nm LED, which is an LED element designed for the peak wavelength of 308 nm, if the peak wavelength of the light emitted from the 308 nm LED is different by only 1 nm due to manufacturing variations, a side effect may occur, or conversely, the therapeutic effect may be insufficient.

For example, let us consider a case in which a person undergoes therapy with an ultraviolet therapy apparatus using a 308 nm LED. The MED for the wavelength of 308 nm of the person is assumed to be 200 mJ/cm². The irradiation dose is set at, for example, 190 mJ/cm² (slightly lower than the MED) in order to prevent a side effect (erythema) and to achieve maximum therapeutic effect. In this case, if the 308 nm LED in the ultraviolet therapy apparatus is actually an LED with a peak wavelength of 307 nm, the affected area will receive ultraviolet irradiation that exceeds the MED for light at the wavelength of 307 nm, which results in erythema in the affected area. Here, the MED for light at the wavelength of 307 nm of the person whose MED for light at the wavelength of 308 nm is 200 mJ/cm² can be calculated to be about 161 mJ/cm² on the basis of the erythema action spectrum Sₑᵣ.

Conversely, if the 308 nm LED in the ultraviolet therapy apparatus is actually an LED with a peak wavelength of 309 nm, the effect on the skin will be less than expected. Therefore, the irradiation of 190 mJ/cm² would not provide the maximum therapeutic effect on the affected area. In other words, a little more irradiation could be applied without causing erythema.

As described above, since the peak wavelength of LEDs can vary depending on the individual LEDs, even though the same ultraviolet irradiation dose is set, the appearance of the side effect and/or the degree of the therapeutic effect may vary depending on therapy apparatuses. In this embodiment, the ultraviolet irradiation dose (the set irradiation dose) set in the ultraviolet therapy apparatus is modified in consideration of such variations in wavelength of the light from the apparatuses.

The inventors have confirmed that although the MED (susceptibility to erythema) varies among individuals for each wavelength, there are no individual differences in a ratio of MED and it conforms to the erythema action spectrum Sₑᵣ defined by the CIE. The ratio of MED is the ratio of the MED for a wavelength to the MED for the wavelength of 308 nm.

FIG. 4 shows the ratio of MED for each individual. The symbols, square, triangle, circle, and lozenge in FIG. 4 are plots of the ratios of MED for four human subjects. The plotted values (ratios of MED) are the MEDs for each wavelength (the MED for 306 nm LED, the MED for 307 nm LED, the MED for 308 nm LED, and the MED for 309 nm LED) divided by the MED for 308 nm LED for each of the human subjects. The dashed curve in FIG. 4 indicates the MED ratio calculated on the basis of the erythema action spectrum Sₑᵣ.

On the basis of the above findings, the inventors have found that on the basis of the ratio of the degree of effect on the human body of reference light emitted from a reference ultraviolet therapy apparatus (reference therapy apparatus) and the degree of effect on the human body of light emitted from an ultraviolet therapy apparatus (subject therapy apparatus) actually used for treatment, the optimal irradiation dose to the patient from the subject therapy apparatus (the irradiation dose from the subject therapy apparatus depending on the patient's MED) can be calculated by modifying the optimal irradiation dose to the patient from the reference therapy apparatus (the irradiation dose of the reference light depending on the patient's MED).

In this embodiment, a case in which an excimer lamp that emits light with a peak wavelength of 308 nm is used for a reference light source is described.

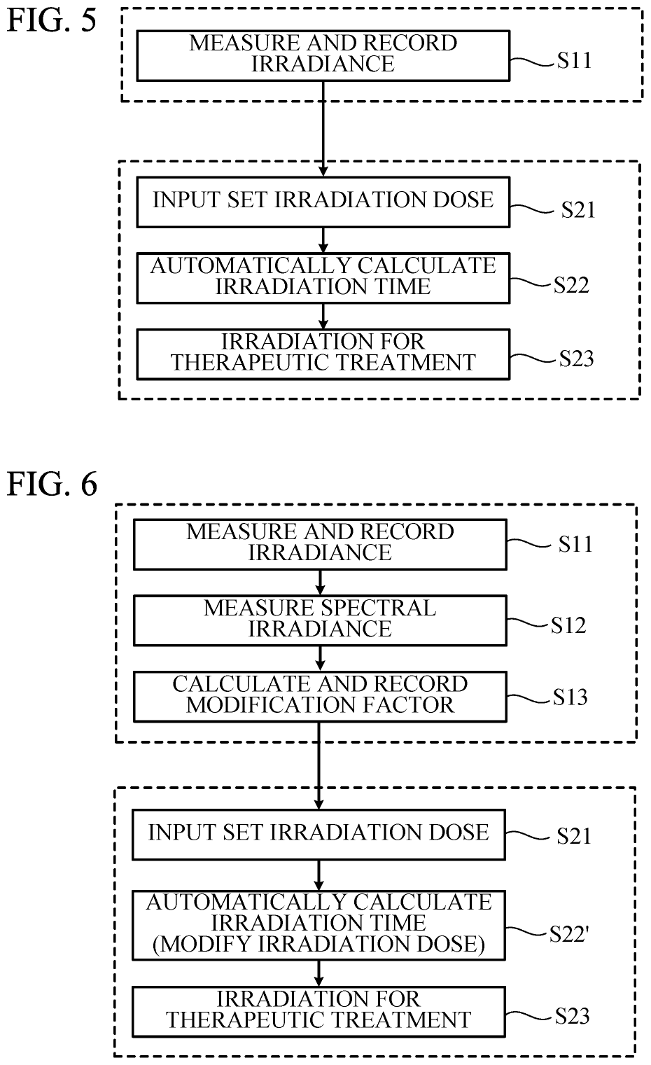

First, a processing flow in a case of using an ultraviolet therapy apparatus having a conventional excimer lamp as a light source is described with reference to FIG. 5.

In contrast to LEDs, excimer lamps have almost no wavelength variation due to manufacturing variations and can emit light with a wavelength of 308 nm, which is targeted for an ultraviolet therapy apparatus. Therefore, there is no need to compensate the amount of ultraviolet irradiation in view of the wavelength difference among the excimer lamps. Here, a case in which an excimer lamp manufactured to emit light of which the wavelength is 308 nm is used for a light source will be described.

In FIG. 5, step S11 is a process to be performed at the factory prior to shipment of the ultraviolet therapy apparatuses, and steps S21 to S23 are processes to be performed during therapy at, for example, a hospital.

At step S11, the irradiance E [mW/cm²] from the ultraviolet therapy apparatus on a light-outputting surface is measured, and the measured irradiance E is stored in the ultraviolet therapy apparatus.

At step S21, a physician examines the patient, determines an appropriate irradiation dose (a set irradiation dose) H [mJ/cm²] for the disease, and inputs the set irradiation dose to the ultraviolet therapy apparatus.

The set irradiation dose H input to the ultraviolet therapy apparatus is the irradiation amount depending on the patient's MED for the light of the excimer lamp at the wavelength of 308 nm. For example, the set irradiation dose H can be slightly less than the patient's MED for the light of the excimer lamp at the wavelength of 308 nm.

At step S22, the irradiation time t is automatically calculated in the ultraviolet therapy apparatus. Specifically, the ultraviolet therapy apparatus obtains the set irradiation dose H input by the physician, divides the set irradiation dose H by the irradiance E stored in the ultraviolet therapy apparatus to calculate the irradiation time t [sec] as in Formula (6).

t=H/E (6)

At step S23, the physician, or in some cases, a nurse or other medical service worker presses the light-outputting surface of the ultraviolet therapy apparatus against the affected area, presses a switch provided in the ultraviolet therapy apparatus to start the ultraviolet irradiation for the therapeutic treatment. The therapeutic treatment is completed after the irradiation time of t seconds.

As described above, when an excimer lamp is used for the light source, the ultraviolet therapy apparatus emits the light with the intended wavelength. In other words, the set irradiation dose H entered at step S21 is the optimum irradiation amount suitable for the patient's MED for the light of the therapy apparatus. Accordingly, the set irradiation dose H can be used for the calculation of the irradiation time t without any modification.

Next, a processing flow when using an ultraviolet therapy apparatus having an LED as a light source according to an embodiment of the present invention will be described with reference to FIG. 6. Here, a case in which an LED manufactured to emit light with a peak wavelength of 308 nm is used for the light source will be described.

As mentioned above, wavelength variation can occur among LED elements. In other words, although LEDs designed to emit light with a wavelength of 308 nm are used for the light source, the peak wavelength of the light emitted from the ultraviolet therapy apparatus may be different from 308 nm. Therefore, the irradiation dose set depending on the patient's MED for light at the wavelength of 308 nm may not be the optimal dose by the therapy apparatus.

Accordingly, in this embodiment, the above-mentioned set irradiation dose H is modified in consideration of the wavelength difference among the LEDs, and ultraviolet irradiation is performed on the basis of the modified set irradiation dose.

In FIG. 6, steps S11 to S13 are processes to be performed at the factory prior to shipment of the ultraviolet therapy apparatuses, and steps S21, S22′, and S23 are processes to be performed during therapy at, for example, a hospital. In FIG. 6, the same step numbers as in FIG. 5 are used to identify the same processes as in FIG. 5.

At step S11, the irradiance E [mW/cm²] from the ultraviolet therapy apparatus on a light-outputting surface is measured, and the measured irradiance E is stored in the ultraviolet therapy apparatus. An ultraviolet therapy apparatus using an LED light source generally includes multiple LEDs (e.g., 5×5 LED array). The irradiance E measured at step S11 is the irradiance of the composite light emitted from the multiple LEDs.

At step S12, the spectral irradiance (ultraviolet irradiance per wavelength) Eλ, [mW/(cm²·nm)] on the light-outputting surface of the ultraviolet therapy apparatus is measured at least in the wavelength range of 250 nm to 400 nm.

At step S13, a modification factor p for modifying the set irradiation dose H is calculated, and the calculated modification factor p is stored in the ultraviolet therapy apparatus. The method of calculating the modification factor p will be described below.

First, the erythemal ultraviolet irradiance I_{CIE} [mW/cm²] is calculated by integrating the product of the spectral irradiance E_{λ}, on the light-outputting surface of the therapy apparatus and the erythema action spectrum Sₑᵣ according to the CIE over the wavelength range from 250 nm to 400 nm as in Formula (2).

Next, the calculated erythemal ultraviolet irradiance I_{CIE} is normalized (divided) by the integral value of the spectral irradiance E_{λ}, of the ultraviolet therapy apparatus over the wavelength range of 250 nm to 400 nm to produce a relative erythemal ultraviolet irradiance Iᵣₑₗ as in Formula (7).

Iᵣₑₗ=I_{CIE}/(∫₂₀₀⁴⁰⁰E_{λ}d_{λ}) (7)

The relative erythemal ultraviolet irradiance Iᵣₑₗ is the degree of effect on the human body caused by the light output from the light-outputting surface of the therapy apparatus and corresponds to the integral value of the product of the relative irradiation intensity of the spectral spectrum and the relative effectiveness of the erythema action spectrum Sₑᵣ shown in FIG. 3 over the wavelength range of 250 nm to 400 nm.

Accordingly, the relative erythemal ultraviolet irradiance Iᵣₑₗ may be calculated directly by integrating the product of the relative irradiation intensity of the spectral spectrum of the light emitted from the therapy apparatus and the relative effectiveness of the erythema action spectrum Sₑᵣ over the wavelength range of 250 nm to 400 nm, instead of calculating the erythemal ultraviolet irradiance I_{CIE} by integrating the product of the spectral irradiance and relative effectiveness of the erythema action spectrum Sₑᵣ over the wavelength range of 250 nm to 400 nm and by normalizing the erythemal ultraviolet irradiance I_{CIE}.

Next, a reference relative erythemal ultraviolet irradiance I_{rel_std} is calculated in the same way for a reference light. The reference relative erythemal ultraviolet irradiance I_{rel_std} is the degree of effect of the reference light on the human body.

For example, the spectral irradiance E_{λ_std} of the reference light is measured in the wavelength range of 250 nm to 400 nm, and a reference erythemal ultraviolet irradiance I_{CIE_std} is calculated by integrating the product of the measured spectral irradiance E_{λ_std} and the relative effectiveness of the erythema action spectrum Sₑᵣ over the wavelength range of 250 nm to 400 nm. Then, the reference erythemal ultraviolet irradiance I_{CIE_std} is normalized by the integral value of the spectral irradiance E_{λ_std} of the reference light over the wavelength range of 250 nm to 400 nm to produce the reference relative erythemal ultraviolet irradiance I_{rel_std}.

Alternatively, the reference relative erythemal ultraviolet irradiance I_{rel_std} may be calculated by integrating the product of the relative irradiation intensity of the spectral spectrum of the reference light and the relative effectiveness of the erythema action spectrum Sₑᵣ over the wavelength range of 250 nm to 400 nm.

Next, the reference relative erythemal ultraviolet irradiance I_{rel_std} is divided by the relative erythemal ultraviolet irradiance Iᵣₑₗ to produce the modification factor p as in Formula (8).

p=I_{rel_std}/Iᵣₑₗ (8)

The modification factor p calculated by Formula (8) is stored in the ultraviolet therapy apparatus.

At step S21, a physician examines the patient, determines an appropriate irradiation dose (a set irradiation dose) H [mJ/cm²] for the disease, and inputs the set irradiation dose to the ultraviolet therapy apparatus.

The set irradiation dose H input to the ultraviolet therapy apparatus is the irradiation amount depending on the patient's MED for the above-mentioned reference light (e.g., a light from an excimer lamp with a wavelength of 308 nm), and can be, for example, a slightly less than the patient's MED for the reference light.

At step S22, the irradiation time t′ is automatically calculated in the ultraviolet therapy apparatus. Specifically, the ultraviolet therapy apparatus obtains the set irradiation dose H input by the physician and calculates a modified set irradiation dose H′ by multiplying the set irradiation dose H by the modification factor p stored in the ultraviolet therapy apparatus, as in the equation below.

H′(=H×p)

Then, the modified set irradiation dose H′ is divided by the irradiance E stored in the ultraviolet therapy apparatus to produce the modified irradiation time t′ [sec] as in Formula (9).

t′=H×p/E (9)

At step S23, the physician or, in some cases, a nurse or other medical service worker presses the light-outputting surface of the ultraviolet therapy apparatus against the affected area, presses a switch provided in the ultraviolet therapy apparatus to start the ultraviolet irradiation for the therapeutic treatment. The therapeutic treatment is completed after the irradiation time of t′ seconds.

Thus, the irradiation dose actually used for the therapeutic treatment can be the modified set irradiation dose H′(=H×p [mJ/cm²]), which is a modification of the set irradiation dose H [mJ/cm²] entered by the physician by taking into account the variation in the wavelength of the LED light sources.

The appearance of the side effect in a case in which the therapeutic treatment is conducted at the modified set irradiation dose H′ by the therapy apparatus according to the embodiment is equivalent to that occurs in a case in which the therapeutic treatment is conducted at the set irradiation dose H by a reference therapy apparatus that emits the above-described reference light. By setting the same reference light for all ultraviolet therapy apparatuses, physicians can concentrate on therapeutic treatment without being aware of the variation in wavelengths although individual therapy apparatuses have different wavelengths.

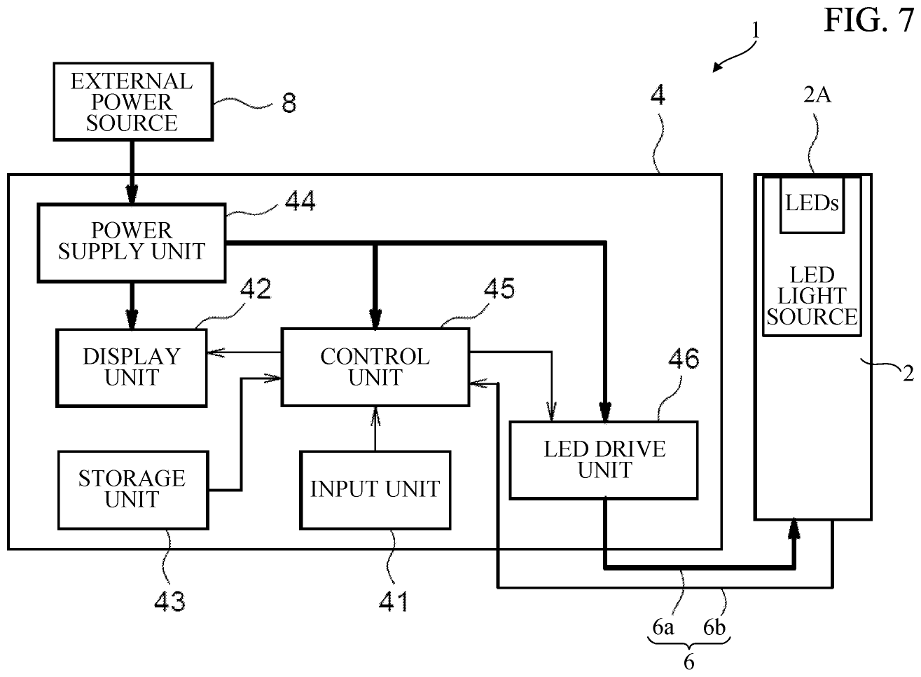

FIG. 7 is a block diagram showing an example configuration of the ultraviolet therapy apparatus 1 according to an embodiment of the present invention.

The ultraviolet therapy apparatus 1 includes a treatment tool (light source part) 2 that has an LED light source that emits light including ultraviolet light and a light-outputting surface 2A through which the light from the LED light source is output, and a main unit 4 that controls the LED light source of the treatment tool 2.

The main unit 4 includes an input unit 41, a display unit 42, a storage unit 43, a power supply unit 44, a control unit (controller) 45, and an LED drive unit 46. The treatment tool 2 and the main unit 4 are connected by a connection cable 6, which has a power line 6a shown by a thick line and a signal line 6b shown by a thin line.

The input unit 41 obtains the set irradiation dose H input by an operator (e.g., the physician) and outputs the information on the set irradiation dose H to the control unit 45.

The display unit 42 can display the UV irradiation intensity, the irradiation time, and the elapsed time during ultraviolet irradiation. The display unit 42 can also display information (such as an error message) indicating that an abnormality has occurred if some abnormality occurs in the ultraviolet therapy apparatus 1.

The storage unit 43 stores the irradiance E on the light-outputting surface of the ultraviolet therapy apparatus 1 and the modification factor p for modifying the set irradiation dose H.

The power supply unit 44 adjusts the voltage of the electric power supplied from an external power source 8 to an appropriate level and supplies the power to each of the units in the subsequent stages.

The control unit 45 modifies the set irradiation dose H input from the input unit 41 with use of the modification factor p stored in the storage unit 43, and calculates the irradiation time t′ by dividing the modified set irradiation dose H′ by the irradiance E stored in the storage unit 43. The control unit 45 then controls the LED drive unit 46 to control the irradiation amount (irradiation time t′) of the LED light source in the treatment tool 2. In other words, the control unit 45 has a modifier that modifies the set irradiation dose H and a lighting controller that causes the LED light source to provide the modified set irradiation dose H′.

The LED drive unit 46 supplies electric power to the LED light source in accordance with the control signal from the control unit 45.

Hereinafter, a procedure in which an operator irradiates ultraviolet to an affected area using the ultraviolet therapy apparatus 1 of the embodiment will be described.

First, the operator manipulates the input unit 41 to input the ultraviolet irradiation dose (set irradiation dose H) for irradiating the affected area. At this time, the ultraviolet therapy apparatus 1 modifies the set irradiation dose H and calculates the ultraviolet irradiation time t′.

Next, the operator holds the treatment tool 2 and brings the light-outputting surface 2A thereof in contact with or close to the affected area. The operator then presses a switch (not shown) provided in the treatment tool 2. The LED light source is then turned on to start the ultraviolet irradiation to the affected area.

Thereafter, when the ultraviolet irradiation amount reaches the modified set irradiation dose H′ (when the irradiation time reaches the calculated irradiation time t′), the LED light source is automatically turned off.

As described above, the ultraviolet therapy apparatus 1 in the embodiment includes a treatment tool (light source part) 2 having an LED light source that emits light including ultraviolet light and a light-outputting surface 2A through which the light from the LED light source is output, and a control unit (controller) 45 that controls lighting of the LED light source. The ultraviolet therapy apparatus 1 also includes an input unit 41 for inputting a set irradiation dose H, which is the irradiation amount of a therapeutic light to be applied to a patient on the assumption that a reference light emitted from a reference light source is used for the therapeutic light, and a storage unit 43 for storing a parameter for modifying the set irradiation dose H.

The storage unit 43 stores, as the parameter, a modification factor p determined on the basis of the degree of effect of the reference light on the human body (the reference relative erythemal ultraviolet irradiance I_{rel_std}) and the degree of effect on the human body caused by the light output from the light-outputting surface of the therapy apparatus (relative erythemal ultraviolet irradiance Lei). The storage unit 43 also stores the irradiance E of the light on the light-outputting surface of the therapy apparatus.

The control unit 45 modifies the set irradiance H on the basis of the modification factor p stored in the storage unit 43 and causes the LED light source to provide the modified set irradiation dose H′. Specifically, the control unit 45 calculates the modified set irradiation dose H′ by multiplying the set irradiation dose H by the modification factor p. The control unit 45 also calculates the irradiation time t′ of the light from the LED light source by dividing the modified set irradiation dose H′ by the irradiance E, and causes the LED light source to emit the ultraviolet for the calculated irradiation time t′.

In other words, a method for applying ultraviolet light using the ultraviolet therapy apparatus 1 in the embodiment includes a first step of inputting a set irradiation dose H, which is the irradiation amount of the therapeutic light to be applied to a patient on the assumption that a reference light emitted from a reference light source is used for the therapeutic light; a second step of modifying the set irradiation dose H by comparing the degree of effect on the human body by the reference light with the degree of effect on the human body by the light output from the light-outputting surface 2A of the therapy apparatus to obtain a modified set irradiation dose of the ultraviolet light; and a third step of causing the LED light source to provide the modified set irradiation dose H′.

Specifically, in the second step, the spectral irradiance E_{λ}, on the light-outputting surface of the therapy apparatus is measured in a predetermined wavelength range. Then, the erythemal ultraviolet irradiance I_{CIE} is calculated by integrating the product of the spectral irradiance E_{λ}, and the relative effectiveness of the erythema action spectrum Sₑᵣ over the wavelength range. Furthermore, the erythemal ultraviolet irradiance I_{CIE} is normalized by the integral value of the spectral irradiance E_{λ}, over the wavelength range to produce the relative erythemal ultraviolet irradiance Iᵣₑₗ, which is the degree of effect on the human body caused by the light output from the light-outputting surface of the therapy apparatus.

In addition, in the second step, the spectral irradiance E_{λ_std} of the reference light is measured in the wavelength range. Then, the reference erythemal ultraviolet irradiance I_{CIE_std} is calculated by integrating the product of the spectral irradiance E_{λ_std} of the reference light and the relative effectiveness of the erythema action spectrum Sₑᵣ over the wavelength range. Furthermore, the reference erythemal ultraviolet irradiance I_{CIE_std} is normalized by the integral value of the spectral irradiance E_{λ_std} of the reference light over the wavelength range to produce the reference relative erythemal ultraviolet irradiance I_{rel_std}, which is the degree of effect of the reference light on the human body.

Then, in the second step, the modification factor p is calculated by dividing the reference relative erythemal ultraviolet irradiance I_{rel_std} by the relative erythemal ultraviolet irradiance Iᵣₑₗ, and the modified set irradiation dose H′ is calculated by multiplying the set irradiation dose H by the modification factor p.

Thus, the LED light source is driven by modifying the set irradiation dose H, which is appropriate when the reference light is used for the therapeutic light, on the basis of the degree of effect on the human body by the reference light (the reference relative erythemal ultraviolet irradiance I_{rel_std}) and the degree of effect on the human body by the light output from the light-outputting surface of the therapy apparatus (relative erythemal ultraviolet irradiance Iᵣₑₗ) Accordingly, if there is a difference in wavelength between the reference light and the light emitted from the therapy apparatus, ultraviolet irradiation can be applied with an irradiation dose that is suitable for the light emitted from the therapy apparatus. Therefore, excellent therapeutic effects can be obtained without causing a side effect to the affected area.

If the reference light is the light emitted from the excimer lamp, which is the light source for the conventional ultraviolet therapy apparatus, physicians can input the set irradiation dose H (without any modification) that was set in the conventional ultraviolet therapy apparatus into the therapy apparatus according to the embodiment. In other words, physicians can use the ultraviolet therapy apparatus in the same way as before, without being aware of variation of wavelength in the light source of the ultraviolet therapy apparatus (whether it is an excimer lamp or an LED light source).

Modifications

In the above-described embodiment, the irradiance E is stored in the storage unit 43 of the ultraviolet therapy apparatus 1, and the control unit 45 calculates the irradiation time t′ of the light from the LED light source by dividing the modified set irradiation dose H′ by the irradiance E, and the LED light source emits the light for the calculated irradiation time t′. However, as long as the LED light source provides the modified set irradiation dose H′, the control unit 45 may control the irradiance of the light (e.g., by controlling the current value of the LED light source) instead of the light irradiation time.

In this case, the control unit 45 calculates the irradiance of the light to be output from the light-outputting surface by dividing the modified set irradiation dose H′ by a predetermined irradiation time, and causes the LED light source to emit the light at the calculated irradiance for the predetermined irradiation time. In other words, in this case, there is no need to store the irradiance E in the storage unit 43.

In the above-described embodiment, the modification factor p is stored in the storage unit 43 of the ultraviolet therapy apparatus 1 and the control unit 45 calculates the modified set irradiation dose H′ by multiplying the set irradiation dose H by the modification factor p. However, other parameters for modifying the set irradiation dose H may be stored in the storage unit 43. For example, information necessary for calculating the modification factor p that includes spectral parameters (or the spectral irradiance) of the reference light, the spectral spectrum (or spectral irradiance) of the light emitted from the therapy apparatus, and the erythema action spectrum Sₑᵣ may be stored in the storage unit 43.

In this case, the control unit 45 calculates the modification factor p on the basis of the information stored in the storage unit 43, and multiplies the set irradiation dose H by the calculated modification factor p to calculate the modified set irradiation dose H′.

In the above-described embodiment, the light with the wavelength of 308 nm is used as the therapeutic light, but the wavelength of the therapeutic light can be set arbitrarily depending on the disease.

In the above-described embodiment, the reference light source that emits the reference light is an excimer lamp, but the reference light may be a light emitted from another lamp such as a fluorescent lamp, a light emitted from an LED, or a light having a desired spectrum shape such as a single line spectrum.

The ultraviolet therapy apparatus of the present invention is not limited to the above-described embodiment, and various changes can be made.

For example, the irradiance may be calculated by integrating the spectral irradiance, or the spectral irradiance may be calculated on the basis of the spectral spectrum and the irradiance.

Alternatively, the erythemal ultraviolet irradiances and/or the modification factor may be calculated from the UV indices without using the spectral irradiances or spectral spectra.

Although a certain embodiment has been described above, the embodiment is exemplary only and is not intended to limit the scope of the present invention. The apparatus and the method described herein may be embodied in forms other than those described above. Also, appropriate omissions, substitutions, and modifications may be made to the above-described embodiment without departing from the scope of the present invention. Forms with such omissions, substitutions and modifications are encompassed in the scope of the claims and their equivalents, and belong to the technical scope of the present invention.

### REFERENCE SYMBOLS

1: Ultraviolet therapy apparatus, 2: Treatment tool, 4: Main unit, 41: Input unit, 42: Display unit, 43: Storage unit, 44: Power supply unit, 45: Control unit, 46: LED drive unit

1. An ultraviolet therapy apparatus, comprising:
a light source part having an LED light source that emits light including ultraviolet light and a light-outputting surface through which the light from the LED light source is output;
a controller configured to control lighting of the LED light source;
an input unit configured to input a set irradiation dose, which is an irradiation amount of a therapeutic light to be applied to a patient on the assumption that a reference light emitted from a reference light source is used for the therapeutic light; and
a storage unit for storing at least one parameter for modifying the set irradiation dose,
the controller including:
a modifier configured to modify the set irradiation dose input by the input unit on the basis of the parameter stored in the storage unit to obtain a modified set irradiation dose of the ultraviolet light, and
a lighting controller configured to cause the LED light source to provide the modified set irradiation dose of the ultraviolet light obtained by the modifier, wherein the modified set irradiation dose is derived from a degree of effect of the reference light on a human body and a degree of effect on the human body caused by the ultraviolet light output from the light-outputting surface.

2. The ultraviolet therapy apparatus according to claim 1, wherein the storage unit stores, as the at least one parameter, a modification factor, which is a value obtained by dividing the degree of effect of the reference light on the human body by the degree of effect on the human body caused by the ultraviolet light output from the light-outputting surface, and
wherein the modifier is configured to calculate the modified set irradiation dose by multiplying the set irradiation dose input by the input unit by the modification factor stored in the storage unit.

3. The ultraviolet therapy apparatus according to claim 1, wherein the storage unit stores, as the at least one parameter, at least a spectral spectrum of the reference light, a spectral spectrum of the ultraviolet light output from the light-outputting surface, and an erythema action spectrum, and
wherein the modifier is configured to
calculate the degree of effect of the reference light on the human body on the basis of a product of a relative irradiation intensity of the spectral spectrum of the reference light and a relative effectiveness of the erythema action spectrum stored in the storage unit,
calculate the degree of effect on the human body caused by the ultraviolet light output from the light-outputting surface on the basis of a product of the spectral spectrum of the ultraviolet light output from the light-outputting surface and the erythema action spectrum stored in the storage unit,
calculate a modification factor, which is a value obtained by dividing the degree of effect of the reference light on the human body by the degree of effect on the human body caused by the ultraviolet light output from the light-outputting surface, and
calculate the modified set irradiation dose by multiplying the set irradiation dose input by the input unit by the modification factor.

4. The ultraviolet therapy apparatus according to claim 1, wherein the storage unit stores an irradiance of the ultraviolet light output from the light-outputting surface, and
wherein the lighting controller is configured to calculate an irradiation time of the ultraviolet light from the LED light source by dividing the modified set irradiation dose by the irradiance of the ultraviolet light output from the light-outputting surface, and to cause the LED light source to emit the ultraviolet light for the irradiation time.

5. The ultraviolet therapy apparatus according to claim 1, wherein the lighting controller is configured to calculate an irradiance of the ultraviolet light to be output from the light-outputting surface by dividing the modified set irradiation dose by a predetermined irradiation time, and to cause the LED light source to emit the ultraviolet light at the irradiance for the predetermined irradiation time.

6. The ultraviolet therapy apparatus according to claim 1, wherein the degree of effect of the reference light on the human body is an integral value of a product of a relative irradiation intensity of a spectral spectrum of the reference light and a relative effectiveness of an erythema action spectrum over a predetermined wavelength range, and wherein the degree of effect on the human body caused by the ultraviolet light output from the light-outputting surface is an integral value of a product of a relative irradiation intensity of a spectral spectrum of the ultraviolet light output from the light-outputting surface and the relative effectiveness of the erythema action spectrum over the predetermined wavelength range.

7. The ultraviolet therapy apparatus according to claim 6, wherein the wavelength range is from 250 nm to 400 nm.

8. The ultraviolet therapy apparatus according to claim 1, wherein the degree of effect of the reference light on the human body is a UV index of the reference light, and wherein the degree of effect on the human body caused by the ultraviolet light output from the light-outputting surface is a UV index of the ultraviolet light output from the light-outputting surface.

9. The ultraviolet therapy apparatus according to claim 1, wherein the LED light source is manufactured to emit the ultraviolet light having a peak wavelength within a range of 308 nm to 313 nm.

10. The ultraviolet therapy apparatus according to claim 9, wherein the LED light source is manufactured to emit the ultraviolet light having a peak wavelength at 308 nm.

11. The ultraviolet therapy apparatus according to claim 1, wherein the reference light source is a lamp that emits the reference light having a peak wavelength within a range of 308 nm and 313 nm.

12. The ultraviolet therapy apparatus according to claim 11, wherein the reference light source is an excimer lamp.

13. The ultraviolet therapy apparatus according to claim 1, wherein the reference light source is an LED light source that emits the reference light having a peak wavelength within a range of 308 nm and 313 nm.

14. A method for applying ultraviolet light using an ultraviolet therapy apparatus having a light-outputting surface through which light including ultraviolet light emitted from an LED light source is output, comprising:
a first step of inputting a set irradiation dose, which is an irradiation amount of a therapeutic light to be applied to a patient on the assumption that a reference light emitted from a reference light source is used for the therapeutic light;
a second step of modifying the set irradiation dose by comparing a degree of effect on a human body by the reference light with a degree of effect on the human body by the ultraviolet light output from the light-outputting surface to obtain a modified set irradiation dose of the ultraviolet light; and
a third step of causing the LED light source to provide the modified set irradiation dose.

15. The method for applying ultraviolet light according to claim 14; wherein the second step includes:
a step of measuring a spectral spectrum of the ultraviolet light output from the light-outputting surface in a predetermined wavelength range;
a step of calculating an erythemal ultraviolet irradiance by integrating a product of a relative irradiation intensity of the spectral spectrum of the ultraviolet light output from the light-outputting surface and a relative effectiveness of an erythema action spectrum over the wavelength range;
a step of calculating a relative erythemal ultraviolet irradiance by normalizing the erythemal ultraviolet irradiance by an integral value of the spectral spectrum of the ultraviolet light output from the light-outputting surface over the wavelength range;
a step of measuring a spectral spectrum of the reference light in the wavelength range;
a step of calculating a reference erythemal ultraviolet irradiance by integrating a product of a relative irradiation intensity of the spectral spectrum of the reference light and the relative effectiveness of the erythema action spectrum of the reference light and the erythema action spectrum over the wavelength range;
a step of calculating a reference relative erythemal ultraviolet irradiance by normalizing the reference erythemal ultraviolet irradiance by an integral value of the spectral spectrum of the reference light over the wavelength range;
a step of calculating a modification factor by dividing the reference relative erythemal ultraviolet irradiance by the relative erythemal ultraviolet irradiance;
a step of calculating the modified set irradiation dose by multiplying the set irradiation dose by the modification factor.
